(19) United States
(12) Patent Application Publication
(10) Pub. No.: US 2025/0262211 A1
(43) Pub. Date: Aug. 21, 2025

(54) COMBINATION OF RAS INHIBITORS AND FARNESYLTRANSFERASE INHIBITORS FOR THE TREATMENT OF CANCERS

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); INSTITUT CLAUDIUS REGAUD, Toulouse (FR); UNIVERSITÉ TOULOUSE III - PAUL SABATIER, Toulouse (FR)

(72) Inventors: Olivier CALVAYRAC, Toulouse (FR); Gilles FAVRE, Toulouse (FR); Célia DELAHAYE, Toulouse (FR)

(21) Appl. No.: 18/856,328

(22) PCT Filed: Apr. 27, 2023
(86) PCT No.: PCT/EP2023/061079

§ 371 Date: Oct. 11, 2024

(30) Foreign Application Priority Data
Apr. 28, 2022 (EP) 22305636.7
Oct. 21, 2022 (EP) 22306602.8

(51) Int. Cl. A61K 31/519; A61K 31/4709; A61P 35/00

(52) U.S. Cl. CPC A61K 31/519; A61K 31/4709; A61P 35/00

(57) ABSTRACT

Lung cancer is the leading cause of cancer deaths worldwide. Metastatic non-small-cell lung cancer (NSCLC) has recently benefited from two consecutive breakthroughs: the identification of oncogene drivers, such as KRAS mutations, leading to the development of targeted therapies, and the understanding of the cancer immunity cycle leading to the development of immune checkpoint inhibitors. KRASG12C mutations can be found in approximately 13% of patients with non-small-cell lung cancer (NSCLC) and historically have been associated with a poor prognosis. Now, data from a phase II trial demonstrate the efficacy of the novel KRASG12C-specific inhibitor sotorasib for patients with advanced-stage NSCLC harbouring this alteration with disease progression on at least one standard-of-care therapy. However one could expect that resistance to Ras inhibitors could occur and that there is a need for identifying new therapeutic avenues for limiting said resistance. The inventors now show that when use alone, sotorasib and tipifarnib did not show a significant anti-tumor effect on lung cancer cells harboring the G12C KRAS mutation, whereas the combination potently induced cell death, suggesting a synergism between these drugs. The present invention thus relates to the combination of Ras inhibitors and famesyltransferase inhibitors for the treatment of cancers.

### FIELD OF THE INVENTION

The present invention is in the field of medicine, in particular oncology.

### BACKGROUND OF THE INVENTION

Lung cancer is the leading cause of cancer deaths worldwide. Metastatic non-small-cell lung cancer (NSCLC) has recently benefited from two consecutive breakthroughs: the identification of oncogene drivers, such as KRAS mutations, leading to the development of targeted therapies, and the understanding of the cancer immunity cycle leading to the development of immune checkpoint inhibitors. KRASG12C mutations can be found in approximately 13% of patients with non-small-cell lung cancer (NSCLC) and historically have been associated with a poor prognosis. Now, data from a phase II trial demonstrate the efficacy of the novel KRASG12C-specific inhibitor sotorasib for patients with advanced-stage NSCLC harbouring this alteration with disease progression on at least one standard-of-care therapy. However one could expect that resistance to Ras inhibitors could occur as for other targeted therapies (e.g. EGFR) and that there is a need for identifying new therapeutic avenues for limiting said resistance. Recently, it has been shown that farnesyltransferase inhibition overcomes the adaptive resistance to osimertinib in EGFR-mutant NSCLC (Sarah Figarol, Célia Delahaye, Rémi Gence, Raghda Asslan, Sandra Pagano, Claudine Tardy, Jacques Colinge, Jean-Philippe Villemin, Antonio Maraver, Irene Ferrer, Luis Paz-Ares, Isabelle Lajoie-Mazenc, Estelle Clermont, Anne Casanova, Anne Pradines, Julien Mazières, Olivier Calvayrac, Gilles Favre; Farnesyltransferase inhibition overcomes the adaptive resistance to osimertinib in EGFR-mutant NSCLC; bioRxiv 2022.04.01.486707; doi: https://doi.org/10.1101/2022.04.01.486707).

### SUMMARY OF THE INVENTION

The present invention is defined by the claims. In particular, the present invention relates to the combination of Ras inhibitors and farnesyltransferase inhibitors for the treatment of cancers.

### DETAILED DESCRIPTION OF THE INVENTION

### Main Definitions

As used herein, the term “Ras” has its general meaning in the art and represents any member of the Ras family of proteins or mutants thereof. Ras family proteins include, but are not limited to, HRAS, KRAS and NRAS, as well as other members of this subfamily as well: DIRAS1; DIRAS2; DIRAS3; ERAS; GEM; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; RRAS2 (Wennerberg et al, The Ras superfamily at a glance, J. Cell. Sci., 2005, 118 (Pt 5), 843-846).

As used herein, the term “mutated-Ras cancer” refers to a cancer in which the cancer cells comprise an activating mutation in a Ras protein.

As used herein, the term “Ras mutation” refers to an activation mutation in a ras gene or Ras protein. A Ras mutation can refer to either a genetic alternation in the DNA sequence of one of the ras genes that results in activation of the corresponding Ras protein, or the alteration in the amino acid sequence of a Ras protein that results in its activation. In particular, the Ras mutation is a KRAS mutation. As used herein, the term “KRAS mutation” includes any one or more mutations in the KRAS (which can also be referred to as KRAS2 or RASK2) gene. For example, the KRAS mutations are located in exon 3 or exon 4 of the gene. Examples of KRAS mutations include, but are not limited to, G12C, G12D, G13D, G12R, G12S, and G12V. KRAS is one of the commonly mutated oncogenes in human cancers. In particular, KRAS mutations are found in 30-40% of tumors and represent together with APC one of the somatic alteration involved in the initiation of colorectal cancer. This mutation occurs early in the process of carcinogenesis, and is maintained at the various stages of disease progression, such as node involvement and metastatic spread. A recent study involving a large number of patients has demonstrated that mutated KRAS is associated with worse outcome in colorectal cancer progression, with effects being more pronounced in stage II and III disease (Nash, et al, Ann. Surg. Oncol, 17:416-424, 2010). The same group has shown, in another study (Nash, et al, Ann. Surg. Oncol, 17:572-578, 2010), that KRAS mutation is associated with more rapid and aggressive metastatic behavior of colorectal liver metastases. In addition, KRAS mutation has been reported to induce drug resistance and treatment failure to epidermal-growth factor receptor (EGFR)-targeting therapeutics in metastatic colorectal cancer. KRAS mutations confer resistance to both cetuximab (Erbitux®) and panitumumab (Vectibix®) (Allegra et al, J. Clin. Oncol, 27:2091-2096, 2008; Linardou et al, Lancet Oncol, 9:962-972, 2008). A number of mutations in NRAS are known and typically include Q61R, Q61K, Q61H, Q61L, Q61N, Q61E, Q61P, A146T, A146P, or A146V.

As used herein, the term “Ras inhibitor” refers to any compound that (i) directly interact with RAS, e.g., by binding to RAS and (ii) decrease the expression or the activity of RAS. In some embodiments, the Ras inhibitor is not tipifarnib. In particular, the refers to an inhibitor of Ras kinase membrane translocation and activity. A Ras inhibitor may be any type of molecule, including, but not limited to, small molecules, antibodies and expression modulators (such as antisense molecules, microRNAs, siRNAs, aptamers, etc.), and may act directly on the Ras protein, may interfere with expression of the Ras protein (e.g., transcription, splicing, translation, and/or post-translational processing), and/or may prevent improper intracellular localization and/or membrane translocation, and/or phosphorylation and/or activation of the Ras protein. Methods of determining whether a compound is a Ras inhibitor are well known (e.g. Haider K, Sharma A, Yar M S, Yakkala P A, Shafi S, Kamal A. Novel approaches for the development of direct KRas inhibitors: structural insights and drug design. Expert Opin Drug Discov. 2022 March; 17 (3): 247-257. doi: 10.1080/17460441.2022.2029842. Epub 2022 Jan. 27. PMID: 35084268.).

As used herein, the term “resistance to Ras inhibitors” is used in its broadest context to refer to the reduced effectiveness of at least one Ras inhibitor to inhibit the growth of a cell, kill a cell or inhibit one or more cellular functions, and to the ability of a cell to survive exposure to an agent designed to inhibit the growth of the cell, kill the cell or inhibit one or more cellular functions. The resistance displayed by a cell may be acquired, for example by prior exposure to the agent, or may be inherent or innate. The resistance displayed by a cell may be complete in that the agent is rendered completely ineffective against the cell, or may be partial in that the effectiveness of the agent is reduced. Accordingly, the term “resistant” refers to the repeated outbreak of cancer, or a progression of cancer independently of whether the disease was cured before said outbreak or progression.

As used herein, the terms “persister cell”, “persister cancer cell”, “drug tolerant persister” and “DTP” are intended to refer to a small subpopulation of cancer cells that maintain viability under anti-cancer targeted therapy treatments, in particular a treatment with a Ras inhibitor. More particularly, it refers to cancer cells that have a tolerance to high concentrations of a treatment of a Ras inhibitor, when it is used in concentrations that are 100 of times higher than IC50. These cells have a slow growth and are almost quiescent.

As used herein, the term “drug-tolerant expanded persister”, or “drug tolerant cells” as used herein, refers to cancer cells that are capable to proliferate with continuous cancer drug treatment in high concentrations, in particular a treatment with a Ras inhibitor.

As used herein, the term “relapse” refers to reappearance of the cancer after an initial period of responsiveness (e.g., complete response or partial response). The initial period of responsiveness may involve the level of cancer cells falling below a certain threshold, e.g., below 20%, 15%, 10%, 5%, 4%, 3%, 2%, or 1%. The reappearance may involve the level of cancer cells rising above a certain threshold, e.g., above 20%, 15%, 10%, 5%, 4%, 3%, 2%, or 1%. More generally, a response (e.g., complete response or partial response) can involve the absence of detectable MRD (minimal residual disease). In some embodiments, the initial period of responsiveness lasts at least 1, 2, 3, 4, 6, 8, 10, or 12 months; or at least 1, 2, 3, 4, or 5 years.

As used herein, the term “farnesyltransferase inhibitor” refers to a molecule that prevents the enzymatically catalysed transfer of a farnesyl residue to a substrate. Herein, the substrate that is farnesylated is typically a polypeptide of at least four amino acids in length. A polypeptide that is enzymatically catalysed farnesylysed preferably includes a CAAX-sequence-motive, at which C represents a cysteine moiety, A an aliphatic amino acid moiety and X another amino acid moiety that is identified by the enzyme that catalyses the farnesylation. As used herein, the enzymatically catalysed transfer of a farnesyl residue describes a biochemical reaction in which a farnesyl residue is transferred to a substrate, preferably a polypeptide. An enzyme that catalyses the transfer of a farnesyl residue to a substrate is called farnesyltransferase. In this case, typically, activated farnesole is transferred. Activated farnesole is preferably farnesyldiphosphate (farnesylpyrophosphate, FPP). Typically, the polypeptide that represents the substrate is farnesylated to a cysteine moiety. So a thiolester is generated. The terms “thiolester” and “thioester” are exchangeable and describe a R1-CO—S—R2 group, wherein a thiolester can also comprise the tautomeric form of the ester R1-COH═S—R2. Preferably, the cysteine moiety that may be farnesylated is localised near to the C-terminal ending of the protein. Particularly preferably, the cysteine moiety of a CAAX-sequence-motive is farnesylated, wherein C represents a cysteine moiety, A an aliphatic amino acid moiety and X another amino acid moiety that is identified by the enzyme that catalyses the farnesylation. The enzyme that catalyses the farnesylation is preferably a farnesyltransferase (FTase), that represents a prenyltransferase with the enzyme-classification-number EC 2.5.1.X, more preferably EC 2.5.1.29, EC 2.5.1.58 or EC 2.5.1.59, even more preferably EC 2.5.1.29 or EC 2.5.1.58. The enzyme typically binds one or several zinc ion(s) (Zn2+). Geranylgeranyltransferase may also be effective as farnesyltransferase in the sense of the invention, because this enzyme is also able to farnesylate particular polypeptides. Every substance or every molecular composition that is able to decelerate or to prevent the enzymatically catalysed farnesylation may be a farnesyltransferase inhibitor. Preferably, a deceleration of the farnesylation rate may be understood as a deceleration of more than 10%, more preferred of more than 25%, even more preferred of more than 50%, even more preferred of more than 75%, even more preferred of more than 80%, even more preferred of more than 90% and most preferred of more than 95% by the addition of the farnesyltransferase inhibitor in an suitable concentration at the site of action compared to a similar reaction environment without addition of the farnesyltransferase inhibitor. More importantly, the farnesyltransferase inhibitor inhibits the farnesylation of RhoB. As used herein, the term “Rho B” has its general meaning in the art and refers to ras homolog gene family, member B that is a protein which in humans is encoded by the RHOB gene.

As used herein, the term “combination” is intended to refer to all forms of administration that provide a first drug together with a further (second, third . . . ) drug. The drugs may be administered simultaneous, separate or sequential and in any order. Drugs administered in combination have biological activity in the subject to which the drugs are delivered. Within the context of the invention, a combination thus comprises at least two different drugs, and wherein one drug is at least a Ras inhibitor and wherein the other drug is a farnesyltransferase inhibitor. In some instance, the combination of the present invention results in the synthetic lethality of the cancer cells, in particular DTC.

As used herein, the expression “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

The terms “kit” or “combined preparation”, as used herein, defines especially a “kit-of-parts” in the sense that the combination partners as defined above can be dosed independently or by use of different fixed combinations with distinguished amounts of the combination partners, i.e. simultaneously or at different time points. The parts of the kit-of-parts can then, e.g., be administered simultaneously or chronologically staggered, that is at different time points and with equal or different time intervals for any part of the kit of parts. The ratio of the total amounts of the combination partners to be administered in the combined preparation can be varied. The combination partners can be administered by the same route or by different routes.

### Methods of the Present Invention

The first object of the present invention relates to a method of treating cancer in a subject in need thereof comprising administering to the subject a therapeutically effective combination comprising a Ras inhibitor and a farnesyltransferase inhibitor.

A further object of the present invention relates to a method delaying and/or preventing development of a cancer resistant to a Ras inhibitor in a subject comprising administering to the subject a therapeutically effective amount of the Ras inhibitor in combination with a farnesyltransferase inhibitor.

A further object of the present invention relates to a method of treating a cancer resistant to a Ras inhibitor in a subject in need thereof comprising administering to the subject a therapeutically effective amount of a farnesyltransferase inhibitor.

A further object of the present invention relates to a method of preventing resistance to an administered Ras inhibitor in a subject suffering from a cancer comprising administering to the subject a therapeutically effective amount of a farnesyltransferase inhibitor.

A further object of the present invention relates to a method for enhancing the potency of a Ras inhibitor administered to a subject suffering from a cancer as part of a treatment regimen, the method comprising administering to the subject a pharmaceutically effective amount of a farnesyltransferase inhibitor in combination with the Ras inhibitor.

A further object of the present invention relates to the use of a farnesyltransferase inhibitor for inhibiting or preventing proliferation of cancer persistent cells or formation of colonies of cancer persistent cells, thereby preventing or delaying the cancer relapse and/or the emergence of acquired resistance to therapies with Ras inhibitors. In addition, this effect against cancer persistent cells may allow to reach a complete response to the cancer treatment. Indeed, the farnesyltransferase inhibitor would be able to eliminate the cancer persistent cells. It also relates to a method for removing or decreasing the cancer persister cell population and/or for preventing or delaying the cancer relapse and/or the emergence of acquired resistance to a cancer treatment, comprising administering a therapeutically effective amount of a farnesyltransferase inhibitor, thereby removing or decreasing the cancer persister cell population. The farnesyltransferase inhibitor would be beneficial in targeting viable persister tumor cells and thus may prevent the emergence of drug-resistant clone(s), in particular in the context of a combined treatment with a Ras inhibitor. The farnesyltransferase inhibitor of the present invention is thus particularly suitable for eradicating drug-tolerant expanded persister.

According to the present invention, the patient suffers from a Ras-mutated cancer. Various cancers are encompassed by the scope of the invention, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testis, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma (including cutaneous or peripheral T-cell lymphoma), Hodgkins lymphoma, non-Hodgkins lymphoma, hairy cell lymphoma, histiocytic lymphoma, and Burkitts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatocarcinoma, breast cancer, colon carcinoma, and head and neck cancer, retinoblastoma, gastric cancer, germ cell tumor, bone cancer, bone tumors, adult malignant fibrous histiocytoma of bone; childhood malignant fibrous histiocytoma of bone, sarcoma, pediatric sarcoma; myelodysplastic syndromes; neuroblastoma; testicular germ cell tumor, intraocular melanoma, myelodysplastic syndromes; myelodysplastic/myeloproliferative diseases, synovial sarcoma.

In some embodiments, the cancer is a solid tumor. For instance, the cancer may be sarcoma and osteosarcoma such as Kaposi sarcome, AIDS-related Kaposi sarcoma, melanoma, in particular uveal melanoma, and cancers of the head and neck, kidney, ovary, pancreas, prostate, thyroid, lung, esophagus, breast in particular triple negative breast cancer (TNBC), bladder, colorectum, liver and biliary tract, uterine, appendix, and cervix, testicular cancer, gastrointestinal cancers and endometrial and peritoneal cancers. Preferably, the cancer may be sarcoma, melanoma, in particular uveal melanoma, and cancers of the head and neck, kidney, ovary, pancreas, prostate, thyroid, lung, esophagus, breast in particular (TNBC), bladder, colorectum, liver, cervix, and endometrial and peritoneal cancers.

In some embodiments, the cancer can be selected from the group consisting of leukemia, lymphoma, sarcoma, melanoma, and cancers of the head and neck, kidney, ovary, pancreas, prostate, thyroid, lung, esophagus, breast, bladder, brain, colorectum, liver, and cervix.

In some embodiments, the cancer can be selected from the group consisting of lung cancer, in particular non-small cell lung cancer, leukemia, in particular acute myeloid leukemia, chronic lymphocytic leukemia, lymphoma, in particular peripheral T-cell lymphoma, chronic myelogenous leukemia, squamous cell carcinoma of the head and neck, advanced melanoma with BRAF mutation, colorectal cancer, gastrointestinal stromal tumor, breast cancer, in particular HER2⁺ breast cancer, thyroid cancer, in particular advanced medullary thyroid cancer, kidney cancer, in particular renal cell carcinoma, prostate cancer, glioma, pancreatic cancer, in particular pancreatic neuroendocrine cancer, multiple myeloma, and liver cancer, in particular hepatocellular carcinoma.

In particular, the subject suffers from a lung cancer. As used herein, the term “lung cancer” has its general meaning in the art and refers to a disease in tissues of the lung involving uncontrolled cell growth, which, in some cases, leads to metastasis. The majority of primary lung cancers are carcinomas of the lung, derived from epithelial cells. The main types of lung cancer are small cell lung carcinoma (SCLC) and non-small cell lung carcinoma (NSCLC). In a particular embodiment, the subject suffers from a non-small cell lung cancer. As used herein, the term “non-small cell lung cancer,” also known as non-small cell lung carcinoma (NSCLC), refers to epithelial lung cancer other than small cell lung carcinoma (SCLC). There are three main sub-types: adenocarcinoma, squamous cell lung carcinoma, and large cell lung carcinoma. Other less common types of non-small cell lung cancer include pleomorphic, carcinoid tumor, salivary gland carcinoma, and unclassified carcinoma. Adenocarcinomas account for approximately 40% of lung cancers, and are the most common type of lung cancer in people who have never smoked. Squamous cell carcinomas account for about 25% of lung cancers. Squamous cell carcinoma of the lung is more common in men than in women and is even more highly correlated with a history of tobacco smoking than are other types of lung carcinoma. There are at least four variants (papillary, small cell, clear cell, and basaloid) of squamous cell carcinoma of the lung. Large cell lung carcinomas are a heterogeneous group of malignant neoplasms originating from transformed epithelial cells in the lung. Large cell lung carcinomas are carcinomas that lack light microscopic characteristics of small cell carcinoma, squamous cell carcinoma, or adenocarcinoma. NSCLC may be categorized using the tumor-nodes-metastasis (TNM) staging system. See Spira J & Ettinger, D. S. Multidisciplinary management of lung cancer, N Engl J Med, 350:382-(2004) (hereinafter Spira); Greene F L, Page D L, Fleming I D, Fritz A G, Balch C M, Haller D G, et al (eds). AJCC Cancer Staging Manual. 6th edition. New York: Springer-Verlag, 2002:167-77 (hereinafter Greene); Sobin L H, Wittekind C H (eds). International Union Against Cancer. TNM classification of malignant tumours. 6th edition. New York: Wiley-Liss (2002) (hereinafter Sobin). Accordingly, in some embodiments, the lung cancer may be stratified into any of the preceding stages (e.g., occult, stage 0, stage IA, stage IB, stage IIA, stage IIB, stage IIIA, stage IIIB or stage IV). More particularly, the subject suffers from a EGFR-mutated NSCLC or an ALK-mutated NSLC as described above.

Non-limiting exemplary Ras inhibitors include, but are not limited to DCAI, as disclosed by Maurer (Maurer et al., 2012), Kobe0065 and Kobe2602, as disclosed by Shima (Shima et al., 2013), HBS 3 (Patgiri et al., 2011), AIK-4 (Allinky), Adagrasib, ARS-3248, AZD4785, and Sotorasib. Preferably, the Ras inhibitor is Sotorasib also known as AMG-510, that is an acrylamide-derived KR1s inhibitor developed by Amgen and that has the IUPAC name of 6-fluoro-7-(2-fluoro-6-hydroxyphenyl)-1-[4-methyl-2-(propan-2-yl) pyridin-3-yl]-4-[(2S)-2-methyl-4-(prop-2-enoyl)piperazin-1-yl]-1H,2H-pyrido[2,3-d]pyrimidin-2-one.

In some embodiments, the farnesyltransferase inhibitor may be an antimetabolite such as, exemplarily, an analogue of farnesole, farnesylphosphate, farnesyldiphosphate or a substrate peptide. The farnesyltransferase inhibitor may also be a molecule with a different structure that may bind into the binding pocket of the peptide substrate or the farnesyldiphosphate. Alternatively, the farnesyltransferase inhibitor may be an allosteric inhibitor.

In some embodiments, the farnesyltransferase inhibitor may have any molecular structure. For example, it may be a peptidic agent, a peptidomimetic or a non-peptidic small-molecular agent. A peptidic agent mostly consists of a peptide. However, the peptide may be conjugated to other molecular structures such as, exemplarily, to an organic, biologically compatible polymer (e.g., polyethylene glycol (PEG), polyethylenimine (PEI), hydroxypropyl methacrylamide (HPMA), to a lipid, an alkyl moiety or to another polypeptide. A peptidomimetic is an agent which molecular structure mimics a peptide. A peptidomimetic may contain, for example, beta-amino acids (1 amino acids), gamma-amino acids (y amino acids) or D-amino acids or it may be made out of these or out of a combination of several thereof. A peptidomimetic may also be conjugated to other molecular structures such as, exemplarily, an organic biologically compatible polymer. A peptidomimetic may also be a retro-inverse peptide. A small molecule agent is a molecule with a molecular weight of less than 1500 Da, preferably less than 1000 Da, even more preferably less than 500 Da. A small molecule agent may also be conjugated to other molecular structures such as, exemplarily, an organic biologically compatible polymer.

In some embodiments, the farnesyltransferase inhibitor is selected from the group consisting of R11577 (Zarnestra, Tipifarnib), SCH66336 (Lonafamib), FTI-277, GGTI-298, BMS-214664, L-778 and L-123.

In some embodiments, the farnesyltransferase inhibitor of the present invention is Tipifarnib.

As used herein, the term “tipifarnib”, also known under the trade name Zarnestra® (J&JPRD), refers to an FTase inhibitor (R)-6-[amino (4-chlorophenyl) (1-methyl-1H-imidazol-5-yl)methyl]-4-(3-chlorophenyl)-1-methyl-2 (1H)-quinolinone (also identified as Rl 15777) having the structure shown below:

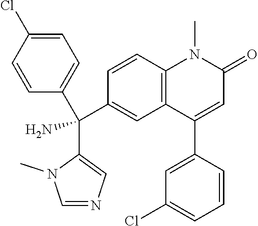 [SMILES: Cn1cncc1[C@@](N)(c1ccc(Cl)cc1)c1ccc2c(c1)c(-c1cccc(Cl)c1)cc(=O)n2C]

Typically, the drug of the present invention is administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

A further object of the present invention relates to a pharmaceutical composition or a kit (kit-of-parts) comprising a Farnesyltransferase inhibitor and a Ras inhibitor, in particular for use for treating cancer.

The invention will be further illustrated by the following figures and examples. However, these examples and figures should not be interpreted in any way as limiting the scope of the present invention.

### FIGURES

FIG. 1. Sotorasib and Tipifarnib synergize for inducing cell death. A. Cell confluency was measured twice a week over 35 days of indicated treatments. B. Amidoblack staining of a 6-well plate after 35 of indicated treatments.

FIG. 2. Tipifarnib prevents the emergence of resistant proliferative clones (RPC) induced by Sotorasib. H23 (A-B) and Calu-1 (C-D) KRAS (G12C)-mutant non-small cell lung cancer (NSCLC) cell lines were transduced with the FUCCI ((Fluorescent Ubiquitination-based Cell Cycle Indicator) system, and response/relapse to sotorasib (1 μM) or sotorasib (1 μM)+tipifarnib (1 μM) was monitored by Incucyte® during 50 days to determine total cell number (A and C) or cell cycle dynamics (B and D).

### EXAMPLE 1

### Methods

H23 cells (KRas G12C) were seeded in a 6-well plate and treated or not with Sotorasib (1 μM), Tipifarnib (1 μM) or the combination. Medium was changed twice a week and cell confluency was monitored by Incucyte® Live-Cell Analysis System.

### Results

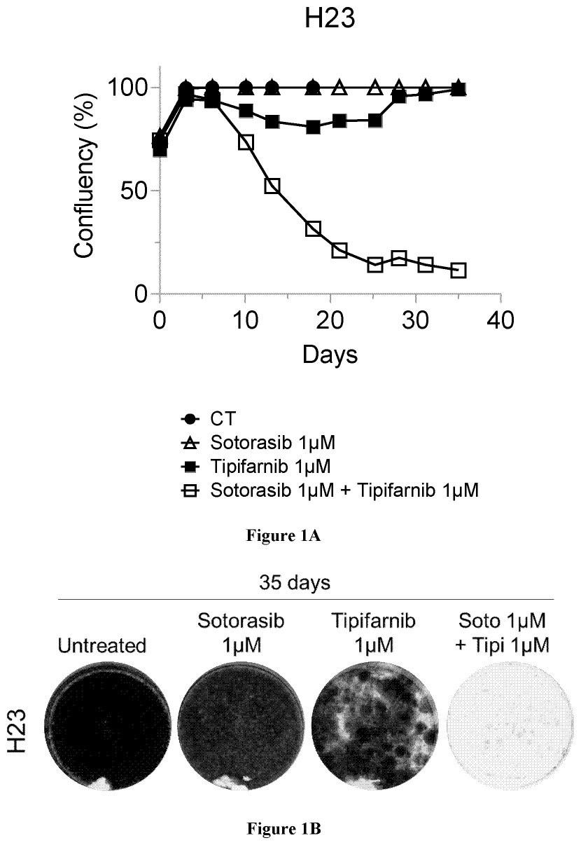

When use alone, sotorasib and tipifarnib did not show a significant anti-tumor effect on H23 cells, whereas the combination potently induced cell death, suggesting a synergism between these drugs (FIG. 1A-B).

### EXAMPLE 2

Method. H23 and Calu-1 KRAS (G12C)-mutant non-small cell lung cancer (NSCLC) cell lines were transduced with the FUCCI (Fluorescent Ubiquitination-based Cell Cycle Indicator) system, and response/relapse to sotorasib (1 μM) or sotorasib (1 μM)+tipifarnib (1 μM) was monitored by Incucyte® during 50 days to determine total cell number or cell cycle dynamics.

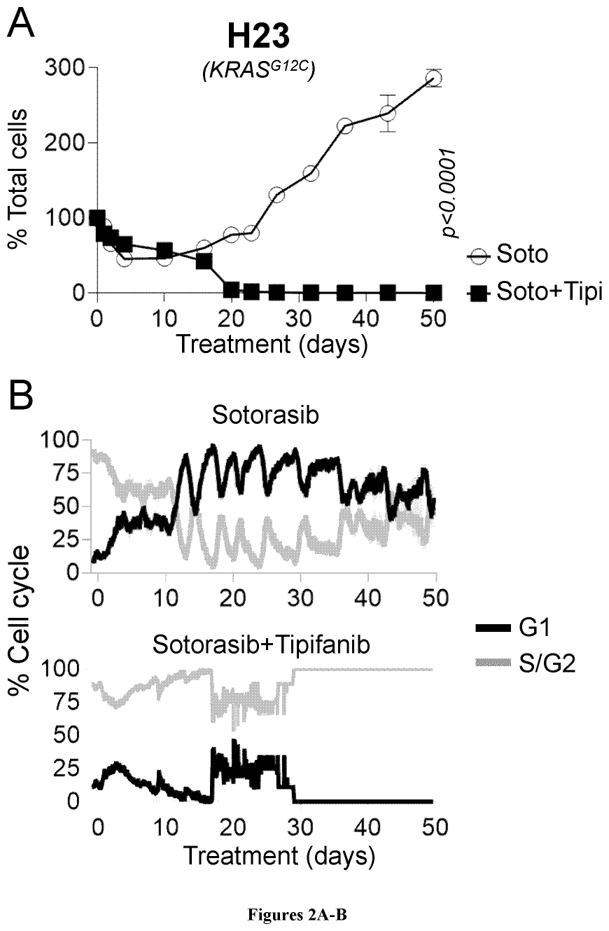

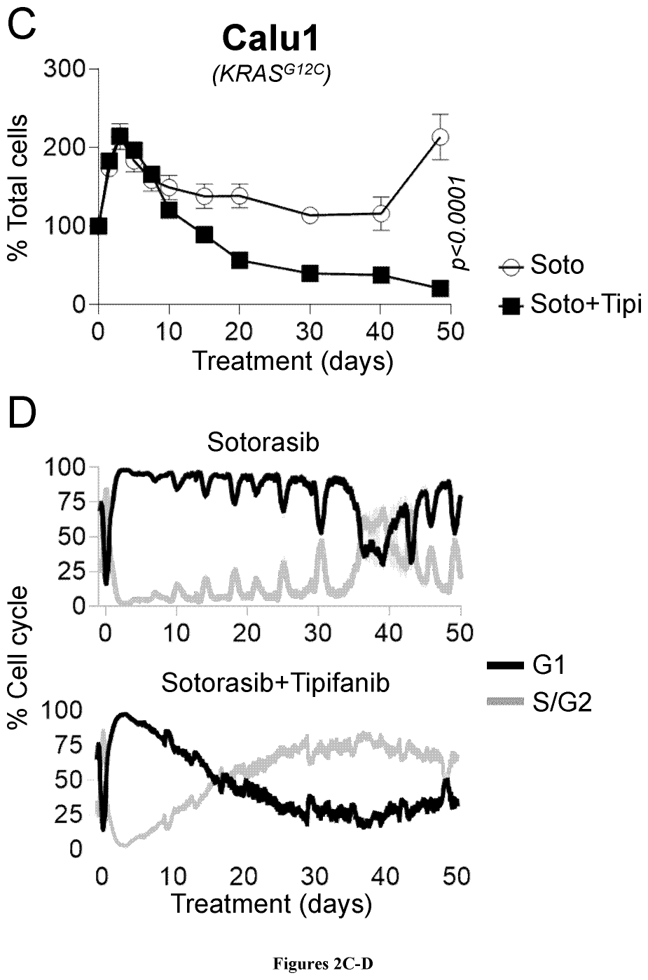

Results. After an initial response to 1 μM sotorasib, H23 and Calu-1 cells developed resistant proliferative clones (RPC) consistent with a gradual increase in the cell cycle dynamics (FIG. 2A, 2B, 2C, 2D). The addition of 1 μM tipifarnib prevented the emergence of RPC by strongly impacting cell cycle dynamics, resulting in cell death (FIG. 2A, 2B, 2C, 2D).

### REFERENCES

Throughout this application, various references describe the state of the art to which this invention pertains. The disclosures of these references are hereby incorporated by reference into the present disclosure.

1. A method of treating cancer in a subject in need thereof comprising administering to the subject a therapeutically effective combination comprising a Ras inhibitor and a farnesyltransferase inhibitor.

2. A method delaying and/or preventing development of a cancer resistant to a Ras inhibitor in a subject comprising administering to the subject a therapeutically effective amount of the Ras inhibitor in combination with a farnesyltransferase inhibitor.

3. A method of treating a cancer resistant to a Ras inhibitor and/or preventing resistance to an administered Ras inhibitor in a subject in need thereof comprising administering to the subject a therapeutically effective amount of a farnesyltransferase inhibitor.

4. (canceled)

5. The method of claim 1, wherein the farnesyltransferase inhibitor inhibits or prevents proliferation of cancer persistent cells, thereby preventing or delaying a cancer relapse and/or emergence of acquired resistance to therapies with Ras inhibitors.

6. The method according to claim 1, wherein the subject suffers from a mutated-Ras cancer.

7. The method of claim 6 wherein the subject harbors a G12C KRAS mutation.

8. The method according to claim 1, wherein the Ras inhibitor is sotorasib.

9. The method according to claim 1, wherein the cancer is selected from the group consisting of leukemia, lymphoma, sarcoma, melanoma, and cancers of the head and neck, kidney, ovary, pancreas, prostate, thyroid, lung, esophagus, breast, bladder, brain, colorectum, liver, and cervix.

10. The method of claim 9 wherein the subject suffers from a non-small cell lung cancer.

11. The method according to claim 1, wherein the farnesyltransferase inhibitor is tipifarnib.

12. A pharmaceutical composition or a kit comprising a farnesyltransferase inhibitor and a Ras inhibitor for use for treating cancer.
